(19) United States
(12) Plant Patent Application Publication
(10) Pub. No.: US 2016/0219766 P1
(43) Pub. Date: Jul. 28, 2016

(54) Apple tree named 'KIRAMEKI'

(50) ;

(71) Applicant: Kazuo YOSHIIE, Nagano (JP)

(72) Inventor: Kazuo YOSHIIE, Nagano (JP)

(21) Appl. No.: 14/998,618

(22) Filed: Jan. 21, 2016

Related U.S. Application Data
(60) Provisional application No. 62/107,180, filed on Jan. 23, 2015.

(51) Int. Cl. A01H 5/00

(52) U.S. Cl. PLT/161

(57) ABSTRACT

A new and distinct apple variety is described, the variety results from controlled cross pollination between ((Jonathan×Pink Pearl)×Fuji). This variety can be characterized by its full golden coloration sometimes with a light blush with very limited over color and intense red fleshed appearance. The new variety has been named ‘Kirameki’.

### CROSS REFERENCE TO RELATED APPLICATION

This application claims the benefit of U.S. Provisional Application No. 62/107,180 filed Jan. 23, 2015, the disclosure of which is incorporated by reference in its entirety.

### GENUS AND SPECIES PLANT CLAIMED

Malus domestica

### VARIETY DENOMINATION

‘Kirameki’

### BACKGROUND OF THE INVENTION

The present invention relates to a new and distinct variety of Malus domestica hereinafter referred to by the cultivar name ‘Kirameki’. The new plant variety described herein resulted from controlled cross pollination between (Jonathan ×Pink Pearl) ×Fuji. The new variety was determined to be distinct from the parent varieties.

### SUMMARY OF THE INVENTION

This variety can be characterized by its full golden coloration sometimes with a light blush with very limited over color and intense red fleshed appearance. The variety is also different in that the harvest time is similar to that of Fuji and flowers early. The new variety has been named ‘Kirameki’. ‘Kirameki’ was asexually reproduced and is currently grown in Japan under Japanese conditions.

### BRIEF DESCRIPTION OF THE DRAWINGS

The accompanying colour photographs show typical specimens of the tree, foliage and fruit of the new variety as depicted in colours as nearly true as is reasonably possible to make the same in a colour illustration of this character.

FIG. 1 shows fruit and foliage on the tree

FIG. 2 shows a close up view of fruit and foliage on the tree

FIG. 3 shows uncut fruit and transverse sections through fruit.

FIG. 4 shows a close up view of uncut fruit, and transverse and longitudinal sections through fruit.

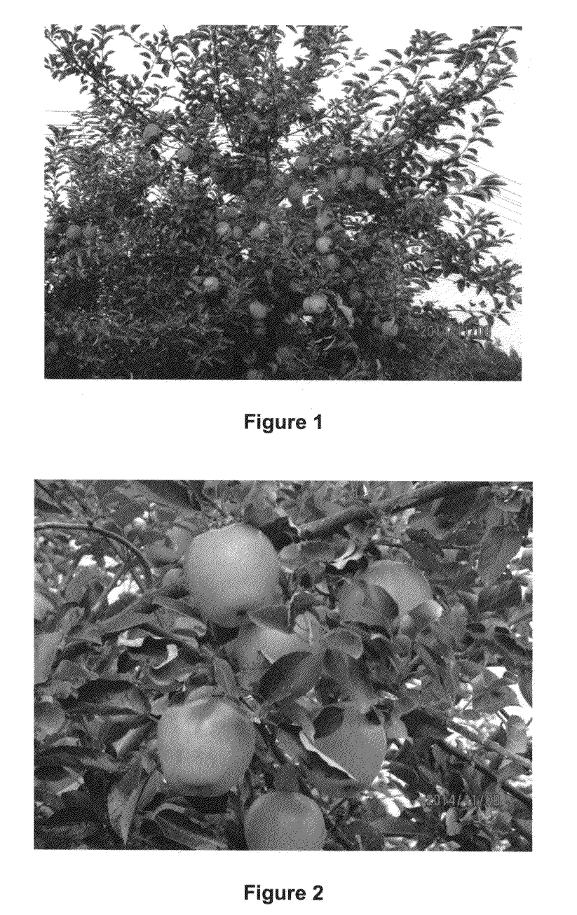

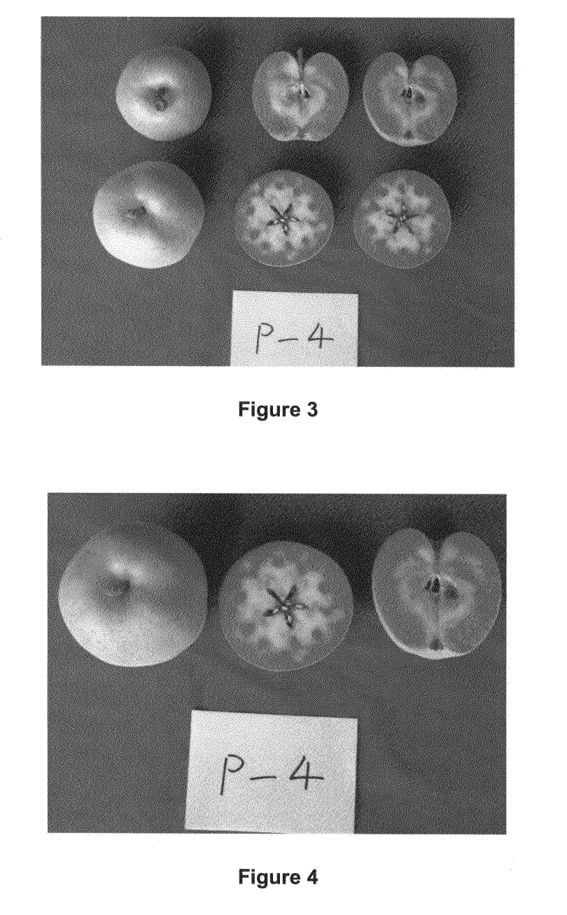

### DETAILED DESCRIPTION

The following is a detailed description of the new variety:
Plant description:
   Breeding.—produced by controlled pollination. Parents: (Jonathan×Pink Pearl)×Fuji. Method of propagating the variety: vegetative propagation through grafting.
   Tree type.—ramified. Tree habit: upright.
Foliage description:
   Length of internode (one-year-old shoot).—short (2.6 cm). Number of lenticels (one-year-old shoot): medium.
   Leaf blade attitude in relation to shoot.—Upwards. Leaf blade length: medium (10.8 cm). Leaf blade width: medium (5.6 cm). Leaf blade ratio length/width: medium (1.93).
   Petiole length.—medium (2.7 cm).
Flower description:
   Predominant color at balloon stage.—dark pink. Diameter with petals pressed into horizontal position: small (3.5 cm). Arrangement of petals: free. Time of beginning of flowering (10% open flowers): early.
Fruit:
   Fruit size.—large (300 g). Height: tall (8.7 cm). Diameter: large (8.6 cm). Ratio height/diameter: large (1.01). Size of eye: small. Bloom of skin: absent or weak. Area of russet around stalk attachment: absent or small. Area of russet around eye basin: absent or small.
   Length of stalk.—medium (2.1 cm) Thickness of stalk: medium (2.3 mm). Depth of stalk cavity: deep (1.7 cm). Width of stalk cavity: medium (4.0 cm). Depth of eye basin: shallow (0.3 cm). Width of eye basin: medium (1.7 cm). Aperture of locules (in transverse section): moderately open.
   General shape of fruit.—obloid. Relative area of over colour of fruit: absent or very small (Granny Smith). Time of eating maturity: late (Fuji). Color of flesh: pinkish red. Acidity of flesh is low.
   Duration of storage.—common and cold storage is very long.

We claim:

1. A new and distinct apple tree substantially as illustrated and described herein.
